(19) United States
(12) Patent Application Publication
(10) Pub. No.: US 2003/0212048 A1
(43) Pub. Date: Nov. 13, 2003

(54) Pharmaceutical compositions comprising in combination a bisphosphonate and a matrix metalloproteinase inhibitor

(76) Inventors: Francois-Regis Bataille, La Chapelle sur Erdre (FR); Martine Laurence Amiot, Nantes (FR)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/444,212

(22) Filed: May 23, 2003

Related U.S. Application Data
(63) Continuation of application No. 09/970,632, filed on Oct. 4, 2001.
Application No. 09/970,632 is a continuation of application No. PCT/EP00/03029, filed on Apr. 5, 2000.

(30) Foreign Application Priority Data
Apr. 7, 1999 (GB) 9907908.9

(51) Int. Cl. A61K 31/675; A61K 31/66

(52) U.S. Cl. 514/89; 514/102

(57) ABSTRACT

A pharmaceutical composition is provided for treatment of malignancies which comprises in combination a bisphosphonate and a matrix metalloproteinase inhibitor for simultaneous, sequential or separate use, provided that the bisphosphonate is not ibandronate when the MMP inhibitor is TIMP-2, e.g. for inhibiting the development of soft tissue metastases during the treatment of a malignant disease with a bisphosphonate wherein treatment with the bisphosphonate gives rise to increased MMP secretion.

This invention relates to pharmaceutical compositions and uses, in particular to pharmaceutical compositions for use in the prophylaxis and treatment of malignant diseases, especially malignant diseases which are associated with the development of bone metastases or excessive bone resorption.

Bisphosphonates have recently become available for longterm treatment of patients with Multiple Myeloma (MM). These pyrophosphate analogs not only reduce the occurrence of skeletal related events but they also provide patients with clinical benefit and improve survival Bisphosphonates are able to prevent bone resorption in vivo; the therapeutic efficacy of bisphosphonates has been demonstrated in the treatment of Paget's disease of bone, tumor-induced hypercalcemia and, more recently, bone metastasis and multiple myeloma (MM) (for review see Fleisch H 1997 Bisphosphonates clinical. In Bisphosphonates in Bone Disease. From the Laboratory to the Patient. Eds: The Parthenon Publishing Group, New York/London pp 68-163). The mechanisms by which bisphosphonates inhibit bone resorption are still poorly understood and seem to vary according to the bisphosphonates studied. Bisphosphonates have been shown to bind strongly to the hydroxyapatite crystals of bone, to reduce bone turn-over and resorption, to decrease the levels of hydroxyproline or alkaline phosphatase in the blood, and in addition to inhibit both the activation and the activity of osteoclasts.

MM is a plasma-cell malignancy characterized by the proliferation and the accumulation of malignant plasma cells within the bone marrow. The main clinical consequences are lytic bone lesions associated with pathologic fractures and bone pain. These lesions result from an excessive bone resorption, frequently leading to hypercalcemia. Bisphosphonates have been introduced for the long-term treatment of MM in combination with conventional chemotherapy. It has been shown recently that bisphosphonates such as clodronate and pamidronate can reduce the occurrence of skeletal related events such as lytic bone lesions and pathologic fractures and can relieve bone pain and improve the quality of life of patients.

The tumour environment and in particular bone marrow stromal cells (BMSC) play an important role both in MM pathogenesis and resistance to treatment such as dexamethasone. It has been previously demonstrated that IL-6, the most potent survival and growth factor for myeloma cells, largely comes from this tumour cell environment, especially from BMSC induced to produce IL-6 in response to myeloma cells according to a paracrine loop. Moreover, we have recently shown that matrix metalloproteinases (MMPs), which play a critical role in bone remodeling and tumor invasion, are also involved in MM pathogenesis. MMP-1 is the only protease known to initiate degradation of type 1 collagen at neutral pH. Collagen 1 is the major component of bone matrix and its degradation by MMP-1 constitutes the initial step of bone resorption. After degradation by MMP-1, denatured type I collagen becomes a substrate for MMP-2. Moreover, both MMP-2 and MMP-9 (i.e., gelatinases A and B respectively) are responsible for collagen IV degradation, the major component of basal membranes, and are involved in tumor dissemination. BMSC secrete MMP-1 (interstitial collagenase) and MMP-2 (gelatinase A). In addition, we have shown that malignant plasma cells can activate the pro-MMP-2 into MMP-2 and can also upregulate the production of MMP-1 in a co-culture system-between BMSC and myeloma cells.

Teronen et al. (U.S. Pat. No. 5,652,227) have described their finding that bisphosphonates have a marked inhibitive effect on MMPs and have proposed that bisphosphonates, including clodronate, etidronate, pamidronate and alendronate, be used as inhibitors of MMPs in methods of treatment to reduce a pathological excess of mammalian collagenolytic enzyme activity and an excessive degradation of connective tissue matrix protein in patients. Among other diseases it is suggested that bisphosphonates may be used to treat excessive degradation of connective tissue matrix protein in cancer and metastasis;progression in connective tissue.

In most cases cancer shows a selective, nonrandom pattern of metastasis to particular organs depending on the site where the primary tumour occurs. Thus breast and prostate cancers are known to have a strong predilection for spreading to bone.

Recently Stearns and Wang (Invasion Metastasis 1996;16: 116-131) have described studies of the combined influence of alendronate and taxol on the establishment and growth of human PC-3 ML prostatic cancer subclones injected intravenously via the tail vein in SCID mice. Pretreatment of SCID mice with alendronate partially blocked the establishment of bone metastases and resulted in tumour formation in the peritoneum and other soft tissues; whereas alendronate pretreatment and dosing along with taxol blocked the growth of PC-3 ML tumours in the bone marrow and soft tissues. The effect of ibandronate and taxol on MMP levels was determined by zymography and ELISA studies with antibodies specific for MMP-2 and it was found that alendronate and taxol independently reduced, in part, the production and secretion of protease by PC-3 ML cells both in vitro and in vivo, and that alendronate in combination with taxol totally blocked MMP-2 production and secretion.

Yoneda et al. have reported a study (J Clin Invest 99:2509-2517) using an experimental model in which inoculation of human estrogen-independent breast cancer MDA-231 cells into the left cardiac ventricle of female nude mice causes osteolytic lesions in bone. To inhibit cancer invasiveness, the tissue inhibitor of the MMP-2 (TIMP-2), which is a natural inhibitor of MMPs, was overexpressed in MDA-231 cells. To inhibit resorption, ibandronate was daily administered subcutaneously. Nude mice received either; (a) nontransfected MDA-231 cells; (b) nontransfected MDA-231 cells and ibandronate; (c) TIMP-2-transfected MDA-231 cells;-or (d) TIMP-2-transfected MDA-231 cells and ibandronate. In mice from group (a), radiographs revealed multiple osteolytic lesions. However, mice from group (b) or group (c), osteolytic lesions were markedly decreased. In animals from group (d) receiving both ibandronate and TIMP-2-transfected MDA-231 cells, there were no radiologically detectable osteolytic lesions.

We have studied the effects of the bisphosphonates, pamidronate and zoledronate, on production of MMPs by IL-1β-stimulated BMSC and have found that zoledronate, but not pamidronate, inhibits MMP-1 production by these cells. However, most surprisingly we have also found that pamidronate and more especially zoledronate are responsible for an upregulation of MMP-2 secretion by BMSC. Thus, totally contrary to previous indications, treatment with bisphosphonates can lead to increase rather than a reduction in the levels of secreted MMPs, in particular MMP-2, with a concomitant increase in the risk of tumour cell dissemination and metastases.

Accordingly the present invention provides a pharmaceutical composition for treatment of malignancies which comprises in combination a bisphosphonate and a matrix metalloproteinase inhibitor for simultaneous, sequential or separate use, provided that the bisphosphonate is not ibandronate when the MMP inhibitor is TIMP-2.

Further the invention provides the use of a MMP inhibitor for the preparation of a medicament, for use in combination with a bisphosphonate for treatment of a malignant disease, for use in inhibiting MMP activity caused by the bisphosphonate, provided that the bisphosphonate is not ibandronate when the MMP inhibitor is TIMP-2.

In a further aspect the invention provides a method of treating a patient suffering from a malignant disease comprising administering to the patient an effective amount of a bisphosphonate and an amount of a MMP inhibitor effective to inhibit MMP activity caused by the bisphosphonate, provided that the bisphoshonate is not ibandronate when the MMP inhibitor is TIMP-2.

Yet further the invention provides use of a MMP inhibitor to inhibit MMP activity caused by a bisphosphonate when the bisphosphonate is used for treatment of a malignant disease, provided that the bisphoshonate is not ibandronate when the MMP inhibitor is TIMP-2.

In the present description the term “treatment” includes both prophylactic or preventative treatment as well as curative or disease modifying treatment, including treatment of patients at risk of contracting the disease or suspected to have contracted the disease as well as in patients.

The invention is generally applicable to the treatment of malignant diseases for which bisphosphonate treatment is indicated. Thus typically the disease is a malignant disease which is associated with the development of bone metastases or excessive bone resorption. Examples of such diseases include cancers,such as breast and prostate cancers, multiple myeloma (MM), tumour induced hypertension (TIH) and similar diseases and conditions. In particular the invention is applicable to the treatment of bone metastases (BM) associated with cancers such as breast cancer.

The compositions, uses and methods of the present invention represent an improvement to existing therapy of malignant diseases in which bisphosphonates are used to prevent or inhibit development of bone metastases or excessive bone resorption, and in which (as has been discovered in accordance with the present invention) bisphosphonate treatment gives rise to an increase in the levels of one or more secreted MMP enzymes, e.g. MMP-2. Use of an MMP inhibitor in combination with the bisphosphonate conveniently inhibits the MMP activity which has been produced in response to treatment with the bisphosphonate, as well as elevated MMP levels, if any, associated with the malignant disease. Advantageously the overall inhibition of MMP activity together with the inhibition of development of bone metastases or excessive bone resorption achieved by the combined MMP inhibitor/bisphosphonate treatment of the present invention leads to an improved treatment outcome and quality of life for patients. In particular the inhibition of MMP activity leads to a lower incidence or less severe occurrence of metastases to soft tissues, which preferably leads to improved patient survival rates and/or a decreased requirement for additional treatment, such as chemotherapy or other cytotoxic treatment.

Thus in preferred embodiments the invention provides:

a) a pharmaceutical composition for inhibiting the development of soft tissue metastases during the treatment of a malignant disease comprising in combination a bisphosphonate and a MMP inhibitor for simultaneous, sequential or separate use, wherein treatment with the bisphosphonate gives rise to increased MMP secretion;

b) use of a MMP inhibitor in the preparation of a medicament to inhibit the development of soft tissue metastases during the treatment of a malignant disease with a combination of a bisphosphonate and a MMP inhibitor, wherein treatment with the bisphosphonate gives rise to increased MMP secretion; or

c) a method of inhibiting the development of soft tissue metastases in a patient during the treatment of a malignant disease comprising administering to the patient an effective amount of a bisphosphonate and an effective amount of a MMP inhibitor.

The bisphosphonates used in the pharmaceutical compositions and treatment methods of the present invention are typically those which can give rise to an increase in the levels of one or more secreted MMP enzymes, e.g. MMP-2, at the doses at which the bisphosphonate is used to prevent or inhibit development of bone metastases or excessive bone resorption.

Thus, for example, suitable bisphosphonates for use in the invention may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-ethidene-bisphosphonic acid, e.g. etidronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-phenylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid; 1-hydroxy-2-(3-pyridyl)ethane-1,1-diphosphonic acid (risedronic acid), e.g. risedronate, including N-methyl pyridinium salts thereof, for example N-methyl pyridin iodides such as NE-10244 or NE-10446; 1-(4-chlorophenylthio)methane-1,1-diphosphonic acid (tiludronic acid), e.g. tiludronate; 3-[N-(2-phenylthioethyl)-N-ethylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenyl-aminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); 1-hydroxy-2-(imidazo[1,2-a]pyridin-3-yl)ethane-1,1-diphosphonic acid, e.g. YM 529; and 1,1-dichloromethane-1,1-diphosphonic acid (clodronic acid), e.g. clodronate.

Pharmaceutically acceptable salts are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

Especially preferred pharmaceutically acceptable salts are those where one, two, three or four, in particular one or two, of the acidic hydrogens of the bisphosphonic acid are replaced by a pharmaceutically acceptable cation, in particular sodium, potassium or ammonium, in first instance sodium.

A very preferred group of pharmaceutically acceptable salts is characterized by having one acidic hydrogen and one pharmaceutically acceptable cation, especially sodium, in each of the phosphonic acid groups.

All the bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130.

A particular embodiment of the invention is represented by the use of a bisphosphonic acid derivative which is selected from 3-amino-1-hydroxypropane-1,1-diphosphonic acid, 3-(N,N-di-methylamino)-1-hydroxypropane-1,1-diphosphonic acid; 4-amino-1-hydroxybutane-1,1-diphosphonic acid; 6-amino-1-hydroxyhexane-1,1-diphosphonic acid, 3-(N-methyl-N-n-pentylamino)-1-hydroxypropane-1,1-diphosphonic acid, 1-hydroxy-2-(imidazol-1-yl)ethane-1,1-diphosphonic acid; 1-hydroxy-2-(3-pyridyl)ethane-1,1-diphosphonic acid, and N-methyl pyridinium salts thereof; 1-(4-chlorophenylthio)methane-1,1-diphosphonic acid; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid; 1-(N-phenylaminothiocarbonyl)methane-1,1-diphosphonic acid; 5-benzoyl-3,4dihydro-2H-pyrazole-3,3-diphosphonic acid tetraethyl ester, 1-hydroxy-2-(imidazo[1,2-a]pyridin-3-yl)ethane-1,1-diphosphonic acid; or a pharmaceutically acceptable salt thereof, and any hydrate thereof.

A preferred embodiment of the invention is represented by the use of a bisphosphonic acid derivative which is selected from 3-amino-1-hydroxypropane-1,1-diphosphonic acid; 3-(N,N-di-methylamino)-1-hydroxypropane-1,1-diphosphonic acid; 4-amino-1-hydroxybutane-1,1-diphosphonic acid; 6-amino-1-hydroxyhexane-1,1-diphosphonic acid; 3-(N-methyl-N-n-pentylamino)-1-hydroxypropane-1,1-diphosphonic acid, 1-hydroxy-2-(imidazol-1-yl)ethane-1,1-diphosphonic acid; 1-hydroxy-2-(3-pyridyl)ethane-1,1-diphosphonic acid; 3-[N-(2-phenylthio-ethyl)N-methylamino]-1-hydroxypropane-1,1-diphosphonic acid; 1-hydroxy-3-(pyrrolidin-1-yl)-propane-1,1-diphosphonic acid; 1-hydroxy-2-(imidazo[1,2-a]pyridin-3-yl)ethane-1,1-diphosphonic acid; or a pharmaceutically acceptable salt thereof, and any hydrate thereof.

A very preferred embodiment of the invention is represented by the use of a bisphosphonic acid derivative which is selected from pamidronic acid, alendronic acid, ibandronic acid, 3-(N-methyl-N-n-pentylamino)-1-hydroxypropane-1,1-diphosphonic acid; 1-hydroxy-2-imidazol 1-yl)-ethane-1,1-diphosphonic acid; risedronic acid and tiludronic acid; or a pharmaceutically acceptable salt thereof, and any hydrate thereof.

An especially preferred embodiment of the invention is represented by the use of a bisphosphonic acid derivative which is selected from 1-hydroxy-2-(imidazol-1-yl)ethane-1,1-diphosphonic acid and 3-amino-1-hydroxypropane-1,1-diphosphonic acid, or a pharmaceutically acceptable salt thereof, and any hydrate thereof.

Further the invention relates to the use of 3-amino-1-hydroxypropane-1,1-diphosphonic acid or a pharmaceutically acceptable salt thereof or any hydrate thereof, e.g. pamidronate disodium or pamidronate.

Further the invention relates to the use of 1-hydroxy-2-(imidazol-1-yl)ethane-1,1-diphosphonic acid or a pharmaceutically acceptable salt thereof or any hydrate thereof, e.g. zoledronate.

The MMP inhibitors used in the pharmaceutical compositions and treatment methods of the present invention include both natural and synthetic MMP inhibitors. Thus the MMP inhibitor may comprise a natural tissue inhibitor of metalloproteinase (TIMP) or an MMP inhibiting part or analog thereof, e.g. TIMP-1, TIMP-2, or functional part or analog thereof.

Conveniently also the MMP inhibitor may be a synthetic MMP inhibitor, such as a hydroxamic acid or hydroxamic acid derivative MMP inhibitor. Examples of such inhibitors are described in British Biotechnology published international patent applications WO 98/52910, WO 98/46563, WO/98/24759, WO 98/23588, WO 97/19053, WO 97/03783, WO 97/1950, WO 96/16931, WO 95/19961, WO 95/19956, WO 95/09841, WO 94/21625, WO 94/24140, WO 94/10990, WO 94/02447, WO 94/02446, WO 93/20047, WO 92/13831, WO 90/05719, WO 90/05716; Hoffmann La Roche published patent applications EP 0684240 Al, EP 0575844 A2, EPO 575844 A2 and EPO 497192 A2; Syntex published patent application WO 95/12603; Merck published patent applications WO 97/11936, U.S. Pat. No. 5,403,952, WO 94/07481 and WO 93/14112; Glaxo published application WO 94/00119 and WO 98/38179; Celltech published application Nos. WO 94/25435, WO 94/25434 and WO 93/24475; Immunex published applications WO 95/06031 and WO/96/41624; Glycomed published application WO 95/19965; EPO 606 046 A1 (Ciba); Chiroscience published applns. WO 95/13289 and WO 97/17088; WO 96/02240 and WO 97/43250 (SmithKline Beecham); WO 97/15553 (Sankyo); WO 97/42168 and WO 98/43959 (Zeneca); WO 95/09260, WO 95/04715 and WO97/09066 (Kaiebo); WO 95/09833 and WO 95/09260 (Florida State University); WO 95/15959 (Schering); WO 97/22587, WO 98/14424 and WO 98/42662 (Novartis); WO 97/24117 (Rhone-Poulenc Rorer); WO 97/47599 and WO 98/27069 (Fujisawa); WO 97/48688 and WO 97/49674, WO 98/13340, WO 98/17645 and WO98/31664 (Pharmacia & Upjohn); WO 98/07697, WO 98/30566, WO 98/33768, WO 98/34915, WO 98/34918, EP 0895988 and WO 99/07675 (Pfizer); WO 98/03164, WO 98/39313, WO 98/39315, WO 98/39316, WO 98/39326 and WO 98/39329 (Monsanto); WO 98/08853, WO 98/08827, WO 98/08825, WO 98/08823, WO 98/08822, WO 98/08815 and WO 99/09003(Procter & Gamble); WO 98/09957, WO 98/09940 and WO 98/09934 (Warner Lambert); WO 98/16503, WO 98/16506, WO 98/16514, WO 98/16520, WO 98/37877 and WO 98/38163 (American Cyanamid), WO 98/43963, WO 98/50348 (Agouron); EP877018 and EP 877019 (Hoechst); WO 99/06410 (Amgen).

Preferably the MMP inhibitor is-is-an inhibitor of MMP-2

Particularly preferred MMP inhibitors for use in the present application are Ro 32,3555 (Trocade), MMI 270, BB 2516 (Marimistat), RS 1308030, AG 3340, BAY 12,95666, more particularly MMP inhibitors as described in EPO 606 046 A1 (Ciba), and WO 97/22587, WO 98/14424 and WO 98/42662 (Novartis), e.g. MMP 090 and TNF 484, especially MMI270.

The bisphosphonates and MMP inhibitors (hereinafter referred to as the Agents of the Invention) may be used in the form of isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The Agents of the Invention can also be used in the form of their hydrates or include other solvents used for their crystallisation.

The Agents of the Invention (the bisphosphonate and the MMP inhibitor) are preferably used in the form of pharmaceutical preparations that contain a therapeutically effective amount of active ingredient optionally together with or in admixture with inorganic or organic, solid or liquid, pharmaceutically acceptable carriers which are suitable for adminstration. The bisphosphonate and MMP inhibitor active ingredients may be present in the same pharmaceutical compositions, though are preferably in separate pharmaceutical compositions. Thus the active ingredients may be administered at the same time (e.g. simultaneously) or at different times (e.g. sequentially) and over different periods of time, which may be separate from one another or overlapping.

The pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

Preferably, the pharmaceutical compositions are adapted to oral or parenteral (especially intravenous or transdermal) administration. Intravenous and oral, first and foremost intravenous, adminstration is considered to be of particular importance. Preferably the the bisphosphonate active ingredient is in the form of a parenteral, most preferably an intravenous form. Preferably the MMP inhibitor is in an oral form.

The particular mode of administration and the dosage may be selected by the attending physician taking into account the particulars of the patient, especially age, weight, life style, activity level, hormonal status (e.g. post-menopausal) and bone mineral density as appropriate.

The dosage of the Agents of the Invention may depend on various factors, such as effectiveness and duration of action of the active ingredient, mode of administration, warm-blooded species, and/or sex, age, weight and individual condition of the warm-blooded animal.

Normally the dosage is such that a single dose of each of the bisphosphonate and MMP inhibitor active ingredients from 0.002-3.40 mg/kg, especially 0.01-2.40 mg/kg, is administered to a warm-blooded animal weighing approximately 75 kg. If desired, this dose may also be taken in several, optionally equal, partial doses.

“mg/kg” means mg drug per kg body weight of the mammal—including man—to be treated.

The dose mentioned above—either administered as a single dose (which is preferred) or in several partial doses—may be repeated, for example once daily, once weekly, once every month, once every three months, once every six months or once a year. In other words, the pharmaceutical compositions may be administered in regimens ranging from continuous daily therapy to intermittent cyclical therapy.

Preferably, the bisphonates are administered in doses which are in the same order of magnitude as those used in the treatment of the diseases classically treated with bisphosphonic acid derivatives, such as Paget's disease, tumour-induced hypercalcemia or osteoporosis. In other words, preferably the bisphosphonic acid derivatives are administered in doses which would likewise be therapeutically effective in the treatment of Paget's disease, tumour-induced hypercalcaemia or osteoporosis, i.e. preferably they are administered in doses which would likewise effectively inhibit bone resorption.

Preferably the MMP inhibitors are administered in doses similar to those customarilly used e.g. for cancer treatment, with additional MMP inhibitor sufficient to inhibit MMP activity caused by the bisphosphonate.

Formulations in single dose unit form contain preferably from about 1% to about 90%, and formulations not in single dose unit form contain preferably from about 0.1% to about 20%, of the active ingredient. Single dose unit forms such as capsules, tablets or dragees contain e.g. from about 1 mg to about 500 mg of the active ingredient.

Pharmaceutical preparations for enteral and parenteral administration are, for example, those in dosage unit forms, such as dragées, tablets or capsules and also ampoules. They are prepared in a manner known per se, for example by mans of conventional mixing, granulating, confectioning, dissolving or lyophising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.

Suitable carriers are especially fillers, such as sugars, for example lactose, saccharose, mannitol or sorbitol, cellulose preparations and/or calcium phosphates, for example tricalcium phosphate or calcium hydrogen phosphate, and also binders, such as starch pastes, using, for example, corn, wheat, rice or potato: starch, gelatin, tragacanth, methylcellulose and/or polyvinylpyrrolidone and, if desired, disintegrators, such as the above-mentioned starches, also carboxymethyl starch, crosslinked polyvinylpyrrolidone, agar or alginic acid or a salt thereof, such as sodium alginate. Adjuncts are especially flow-regulating agents and lubricants, for example silicic acid, talc, stearic acid or salts thereof, such as magnesium or calcium stearate, and/or polyethylene glycol. Dragee cores are provided with suitable coatings that may be resistant to gastric juices, there being used, inter alia, concentrated sugar solutions that optionally contain gum arabic, talc, polyvinylpyrrolidone, polyethylene glycol and/or titanium dioxide, or lacquer solutions in suitable organic solvents or solvent mixtures or, to produce coatings that are resistant to gastric juices, solutions of suitable cellulose preparations, such as alkylcellulose phthalate or hydroxypropylmethylcellulose phthalate. Colouring substances or pigments may be added to the tablets or dragee coatings, for example for the purpose of identification or to indicate different doses of active ingredient.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

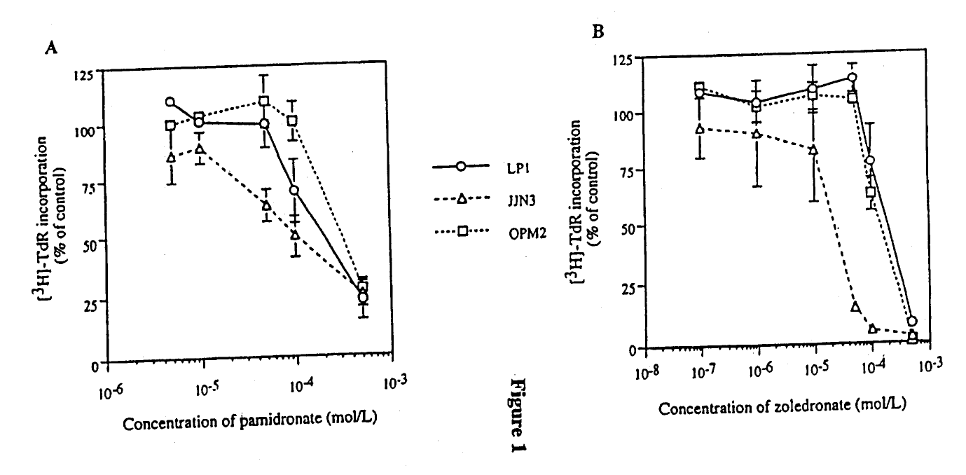

The following Examples illustrate the invention described hereinbefore. The term “active ingredient” is to be understood as being any one of the bisphosphonic acid derivatives or MMP inhibitors mentioned above as being useful according to the present invention. Example 7 refers to the accompanying FIGS. 1-9, in which

FIG. 1 are graphs showing inhibition of myeloma proliferation by bisphosphonates: by (A) pamidronate and (B) by zoledronate, The incorporation of [³H]-TdR being expressed as a percentage of control ±SE;

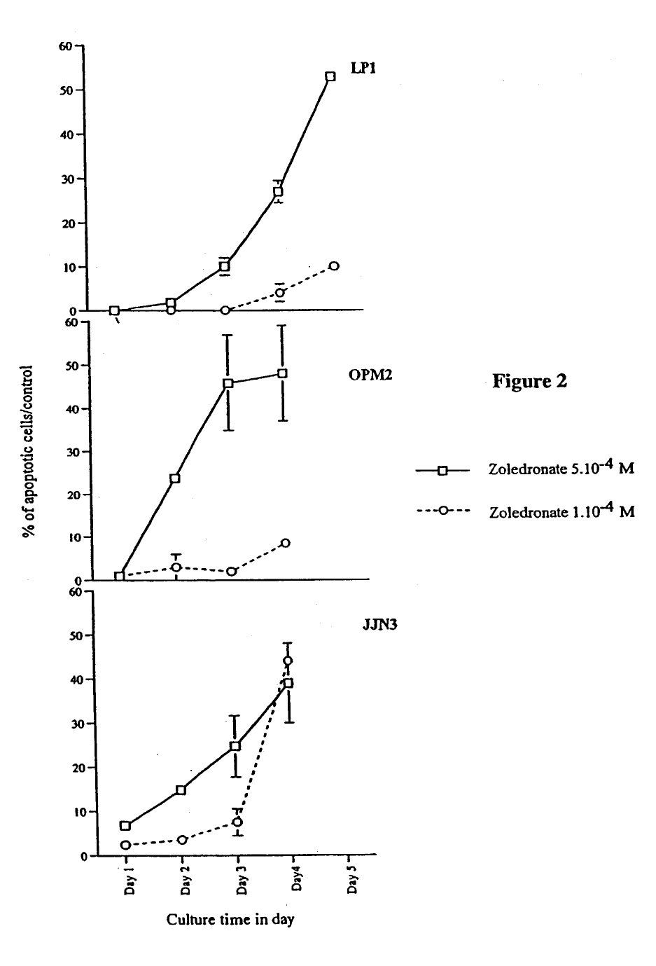

FIG. 2 are graphs showing apoptosis for various HMCL, induce by zoledronate;

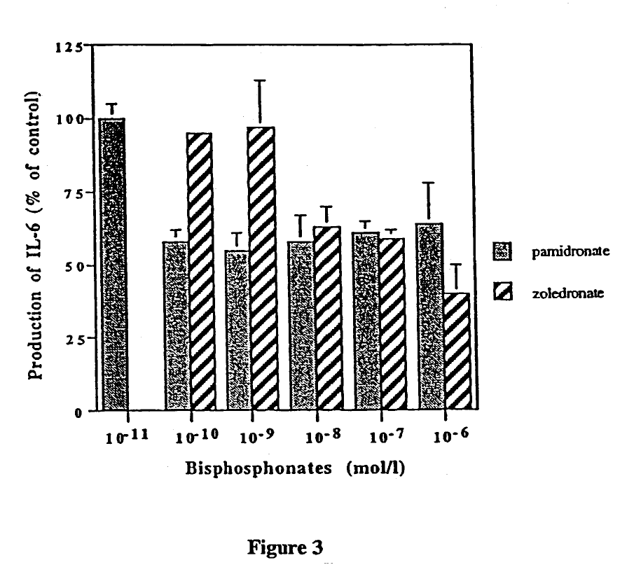

FIG. 3 is a graph showing the effect of either pamidronate or zoledronate on constitutive IL-6 production by BMSC;

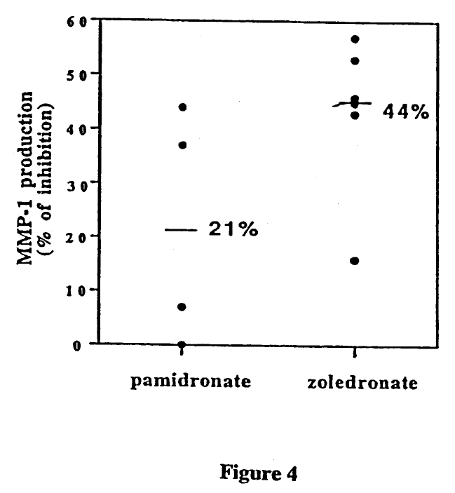

FIG. 4 is a graph showing inhibition of MMP-1 production by BMSC induced either by pamidronate or zoledronate, and

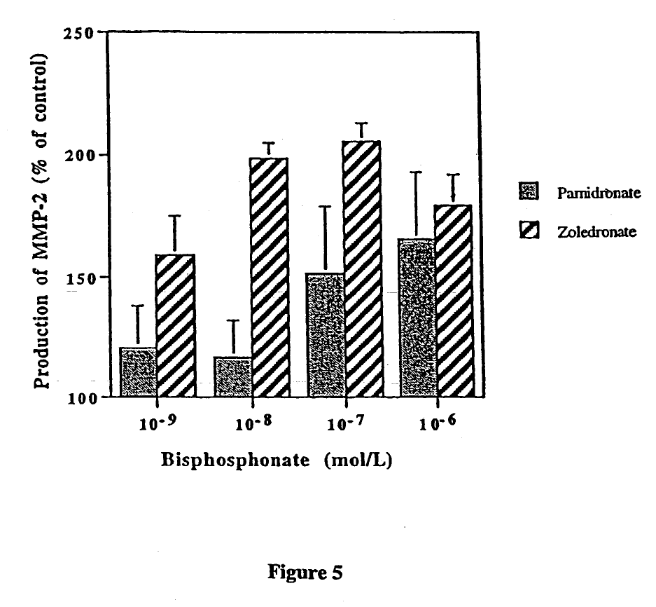

FIG. 5 are graphs showing quantitative analysis of the effects of bisphosphonates on MMP-2 secretion.

### EXAMPLES

### Example 1

Capsules containing coated pellets of active ingredient, for example, disodium pamidronate pentahydrate, as active ingredient:

| | |
|---|---|
| Core pellet: | |
| active ingredient (ground) | 197.3 mg |
| Microcrystalline cellulose | 52.7 mg |
| (Avicel ® PH 105) | |
| | 250.0 mg |
| + Immer coating: | |
| Cellulose HP-M 603 | 10.0 mg |
| Polyethylene glycol | 2.0 mg |
| Talc | 8.0 mg |
| | 270.0 mg |
| + Gastric juice-resistant outer cotaing: | |
| Eudragit ® L 30 D (solid) | 90.0 mg |
| Tirethyl citrate | 21.0 mg |
| Antifoam ® AF | 2.0 mg |
| Water | |
| Talc | 7.0 mg |
| | 390.0 mg |

A mixture of disodium pamidronate with Avicel® PH 105 is moistened with water and kneaded, extruded and formed into spheres. The dried pellets are then successively coated in the fluidized bed with an inner coating, consisting of cellulose HP-M 603, polyethylene glycol (PEG) 8000 and talc, and the aqueous gastric juice-resistant coat, consisting of Eudragit® L 30 D, triethyl citrate and Antifoam® AF. The coated pellets are powdered with talc and filled into capsules (capsule size 0) by means of a commercial capsule filling machine, for example Höfliger and Karg.

### Example 2

Monolith adhesive transdermal system, containing as active ingredient, for example, 1-hydroxy-2-(imidazol-1-yl)-ethane-1,1-diphosphonic acid:

| COmposition: | |
|---|---|
| polyisobutylene (PIB) 300 | 5.0 g |
| (Oppanol B1, BASF) | |
| PIB 35000 | 3.0 g |
| (Oppanol B10, BASF) | |
| PIB 1200000 | 9.0 g |
| (Oppanol B100, BASF) | |
| hydrogenated hydrocarbon resin | 43.0 g |
| (Escorez 5320, Exxon) | |
| 1-dodecylazacycloheptan-2-one | 20.0 g |
| (Azone, Nelson Res., Irvine/CA) | |
| active ingredient | 20.0 g |
| Total | 100.0 g |

Preparation:

The above components are together dissolved in 150 g of special boiling point petroleum fraction 100-125 by rolling on a roller gear bed. The solution is applied to a polyester film (Hostaphan, Kale) by means of a spreading device using a 300 mm doctor blade, giving a coating of about 75 g/m². After drying (15 minutes at 60° C.), a silicone-treated polyester film (thickness 75 mm, Laufenberg) is applied as the peel-off film. The finished systems are punched out in sizes in the wanted form of from 5 to 30cm² using a punching tool. The complete systems are sealed individually in sachets of aluminised paper.

### Example 3

Vial containing 1.0 mg dry, lyophilized 1-hydroxy-2-(imidazol-1-yl)ethane-1,1-diphosphonic acid (mixed sodium salts thereof). After dilution with 1 ml of water, a solution (concentration 1 mg/ml) for i.v. infusion is obtained.

| Composition: | | |
|---|---|---|
| active ingredient (free diphosphonic acid) | 1.0 | mg |
| mannitol | 46.0 | mg |
| Trisodium citrate × 2 H₂O ca. | 3.0 | mg |
| water | 1 | ml |
| water for injection | 1 | ml |

In 1 ml of water, the active ingredient is titrated with trisodium citrate×2 H₂O to pH 6.0. Then, the mannitol is added and the solution is lyophilized and the lyophilisate filled into a vial.

### Example 4

Ampoule containing active ingredient, for instance disodium pamidronate pentahydrate dissolved in water. The solution (concentration 3 mg/ml) is for i.v. infusion after dilution.

| Composition: | | |
|---|---|---|
| active ingredient | 19.73 | mg |
| ( [symbol] 5.0 mg of anhydrous active ingredcient) | | |
| mannitol | 250 | mg |
| water for injection | 5 | ml |

### Example 5

Tablets each containing 50 mg of 3-[N-(2-phenylthioethyl)-N-methylamino]-1-hydroxypropane-1,1-diphosphonic acid or MMI 270 (N-hydroxy-2(R)-[[4-methoxybenzene sulfonyl](3-picolyl)-amino]-3-methylbutanamide hydrochloride) can be prepared as follows:

| Composition (10,000 tablets) | | |
|---|---|---|
| Active ingredient | 500.0 | g |
| Lactose | 500.0 | g |
| Potato starch | 325.0 | g |
| Gelatin | 8.0 | g |
| Talc | 60.0 | g |
| Magnesium stearate | 10.0 | g |
| Silicon dioxide (fuely divided) | 20.0 | g |

| | |
|---|---|
| Ethanol | q.s. |

The active ingredient is mixed with the lactose and 292 g of potato starch, and the mixture is moistened with an ethanolic solution of the gelatin and granulated through a sieve. After the granules have dried, the remainder of the potato starch, the magnesium stearate and the silicon dioxide are admixed and the mixture compressed to give tablets each weighing 145.0 mg and containing 50.0 mg of active ingredient, which can, if desired, be provided with breaking grooves to enable the dosage to be more finely adjusted.

### Example 6

Preparation of 3000 capsules each containing 25 mg of the active ingredient, for example, N-hydroxy-2(R)-[[4-methoxybenzenesulfonyl](3-picolyl)-amino]-3-methylbutanamide hydrochloride:

| | | |
|---|---|---|
| Active ingredient | 75.0 | g |
| Lactose | 750.0 | g |
| Avicel PH 102 | 325.0 | g |
| (microcrystalline cellulose) | | |
| Polyplasdone XL | 30.0 | g |
| (polyvinylpyrrolidone) | | |

| | |
|---|---|
| Purified water | q.s. |

| | | |
|---|---|---|
| Magnesium stearate | 9.0 | g |

The active ingredient is passed through a No. 30 hand screen.

The active ingredient, lactose, Avicel PH 102 and Polyplasdone XL are blended for 15 minutes in a mixer. The blend is granulated with sufficient water (about 500 mL), dried in an oven at 35° C. overnight, and passed through a No. 20 screen.

Magnesium stearate is passed through a No. 20 screen, added to the granulation mixture, and the mixture is blended for 5 minutes in a mixer. The blend is encapsulated in No. 0 hard gelatin capsules each containing an amount of the blend equivalent to 25 mg of the active ingredient.

### Example 7

Experiments on Tumour Cells and Tumour Cell Environment

Materials and Methods

Reagents

Two bisphosphonates are used: pamidronate or (3-amino-1-hydroxypropylidene) bis-phosphonate (APD), which is the reference molecule used in the treatment of MM, and zoledronate or (1-hydroxy-2-(1H-imidazole-1-yl)ethylidene) bisphosphonate, both products of Novartis (Basel, Switzerland). Bisphosphonates are dissolved in PBS (Phosphate Buffer Saline) and stored at −20° C. until use.

Cell Isolation and Culture Conditions

The human myeloma cell lines (HMCL) LP-1 and OPM-2 were obtained from DSM (Braunschweig, Germany). JJN-3 was kindly provided by B Van Camp (VUB, Belgium). Cells are maintained in RPMI 1640 culture medium supplemented with 10% fetal calf serum (FCS), 2 mM glutamine, antibiotics (100 IU/ml penicillin and 100 μg/ml streptomycin) and 10 μM 2-β-mercaptoethanol.

BMSC are obtained from 7 patients with MM after long term culture of bone marrow samples. Bone marrow mononuclear cells are isolated by Ficoll-Hypaque density centrifugation. The cells are plated in DMEM supplemented with 10% FCS and allowed to attach for 3 days, after which the medium is renewed. After 2 to 3 weeks of culture, a confluent adherent cell monolayer is obtained and then, after two passages using trypsin/EDTA solution, BMSC are recovered for study. Cells are maintained in DMEM supplemented with 10% FCS, 2 mmol/L glutamine, 100 μg/mL streptomycin, 100 U/mL penicillin, and 5. 10⁻⁵ mol/L 2-β-mercaptoethanol.

Proliferation of HMCL

The three HMCL are studied i.e., LP-1, OPM-2 and JJN-3. Proliferation assays are performed in 96-well round-bottom microtiter plates at a cell density of 10⁵ cells/mL. Cells are incubated for 3 days at 37° C. in a 5% CO₂ humidified atmosphere with or without recombinant human IL-6 (rhIL-6) at 5 ng/ml, in the presence (or not) of the bisphosphonate of interest. Then, 0,5 MCi of [³H]-thymidine is added during the last 18 hours. [³H]-thymidine, incorporation is quantified by liquid scintillation spectroscopy.

Detection of Apoptosis in HMCL

The presence of apoptotic cells is evaluated after 1 to 5 days of culture of HMCL in the presence or in the absence of bisphosphonates. The percentage of apoptotic cells is determinated by flow cytometry using APO 2.7 monoclonal antibody (mAb) coupled to PE (Immunotech, Marseilles, France) (Zhang et al. 1996, J. Immunol. 157: 3980-3987).

Analysis of Cell Cycle

LP-1 cells (2×10⁵ cells) plated in a 24 well-plate are incubated in the presence of 5×10⁴ Mol/L of either pamidronate or zoledronate. A control without bisphosphonate is performed. Cells are recovered after a 4-day incubation period and washed. They are then incubated for 40 minutes at 37° C. with a solution of Triton X 100 0.1 %, Sodium Citrate 0.1%, 5 IU of RNAse and stained with propidlum iodide (PI) 50 mg/L. Flow cytometry analysis is performed on a FACSCalibur using a CELLQuest program (Becton Dickinson). Data are gated on the F12-Area versus FL2-Width cytogram to exclude doublets and agregates, and a minimum of 2.5×10⁴ gated cells is collected per sample. Analysis of the cell cycle was performed using the Modfit LT for Mac V2.01 program (Verity Software House, Inc). Apoptotic cells were detected as a subdiploid peak as described by Zamai et al. (1993, Cytometry 14: 891-897)

Survival Experiments

Adherent BMSC (3×10⁵ cells) are plated in 25 cm² flasks and are incubated with either pamidronate (APD) or zoledronate at the following concentrations: 10⁻⁴ and 10⁻⁵ mol/L. A control without bisphosphonate is performed. After a 3-day incubation period, cells are washed with PBS and removed from the flasks using trypsin-EDTA for 20 seconds. The percentage of apoptotic cells is determined by Dow cytometry using APO 2.7 mAb (Immunotech, Marseilles, France) (ibid.).

Determination of IL-6 concentrations

Adherent BMSC (10⁴ cells) plated in 96-well plates are preincubated for 24 hours with either pamidronate or zoledronate. The medium is replaced by a fresh one containing 2% FCS and different concentrations of each bisphosphonate: 10^{31 6}, 10⁻⁷, 10⁻⁸, 10⁻⁹, 10⁻¹⁰, 10⁻¹¹ Mol/L of zoledronate and 10^{31 6}, 10^{31 7}, 10⁻⁸, 10⁻⁹, 10⁻¹⁰ Mol/L of pamidronate respectively. After a 48-hour incubation period, the supernatants are collected and stored at −20° C. until analysis. IL-6 production is measured by ELISA (ELISA test concentrations ranging from 10 to 500 pg/mL) (Innotest, Besancon, France). No interference between the bisphosphonates and ELISA is observed in this study (data not shown). Six of the seven BMSC are tested for production of IL-6 in the presence of bisphosphonates.

Determination of interstitial collagenase (MMP-1) levels

Adherent BMSC (15×10³ cells) plated in 96-well plates are preincubated for 24 hours with either pamidronate or zoledronate. Then the cells are stimulated by IL-1β (10 ng/mL) in the presence of different concentrations (10⁻⁶, 10⁻⁷, 10⁻⁴ and 10⁻⁹ Mol/L) of each bisphosphonate without FCS. After a 48-hour incubation period, supernatants are collected and stored at −20° C. until analysis. MMP-1 levels are measured by ELISA (Amersham, Les Ulis, France). This assay measures MMP-1 irrespective of its mode of presentation (i.e. free or bound, active or not).

Determination of gelatinase A (MMP-2) levels by gelatin substrate gel zymography

Adherent BMSC (10⁴ cells) plated in 96-well plates are preincubated for 24 hours with either pamidronate or zoledronate. Then the medium is replaced by a fresh one without FCS but in the presence of different concentrations (10⁶, 10⁻⁷, 10⁻⁸, 10⁻⁹ Mol/L) of each bisphosphonate. After a 48-hour incubation period, the supernatants are collected and stored at −20° C. until analysis. MMP-2 production is shown by gelatin substrate gel zymography. The supernatants are mixed with sodium dodecyl sulfate (SDS) sample buffer without reducing agent and then, proteins are separated by SDS-polyacrylamide gel electrophoresis in 7.5% polyacrylarnide gels containing gelatin at 1 mg/mL, as previously described by Heussen and Dowdle(1980 Ann. Biochem. 102: 196-202) and adjusted for minigel format. After electrophoresis, SDS is removed from the gels by an incubation in 2.5% Triton X-100 for half an hour at room temperature. The gels are then incubated at 37° C. in a buffer containing 50 mM/L Tris-HCl 5 mM/L CaCl₂, pH 7.6 for 24 hours. The gels were stained with Coomassie blue R250 (0.25%). A clear 72 kDa band against the blue background of stained gelatin demonstrated MMP-2 proteolytic activity. The intensity of the gelatinolytic bands is analysed by densitometry. No interference between bisphosphonates and gelatin substrate gel zymography is observed using this assay (data not shown).

Statistical Analysis:

Data are expressed as mean±Standard Deviation. The Wilcoxon signed rank test is used to determine statistical significance of detected differences.

Results

A. Pamidronate and Zoledronate Inhibit the Proliferation of HMCL.

We have investigated the proliferation of three HMCL i.e., LP-1, OPM-2 and JJN-3 in the presence of either pamidronate or zoledronate. Both pamidronate and zoledronate induce an inhibition of proliferation of the 3 HMCL studied. In the presence of 5×10⁻⁴ Mol/L of pamidronate, the percentage of proliferation of the three cell lines is 25% of that of the controL (FIG. 1A). The inhibition of proliferation is dose dependent and observed up to a concentration of pamidronate of 5×10⁻⁵ Mol/L (except for JJN-3 which is more sensitive, needing a concentration of 1×10⁻⁵ Mol/L of pamidronate to return to a proliferation level identical to that of the control). Furthermore, we show that zoledronate is more potent than pamidronate to inhibit proliferation. In the presence of 5×10⁷ Mol/L of zoledronate, an inhibition of proliferation of about 95% is observed (FIG. 1B), versus 75% with pamidronate (FIG. 1A) on all the HMCL. The recovery of a normal prolifertion rate is observed as soon as 5×10⁻⁵ Mol/L of zoledronate for LP-1 and OPM-2 and b 1×10⁻⁵ (Mol/L for JJN-3. The IC₅₀s of the 3 HMCL are very similar in the presence of pamidronate (IC_{50 LP-1}=2.07×10⁻⁴ Mol/L, IC_{50 JJN-3}=1.07×10⁻⁴ Mol/L and IC_{50 OPM-2}=2.93×10⁻⁴ Mol/L). Moreover, the IC₅₀s of the cell lines in the presence of zoledronate (IC_{50 LP-1}=1.93×10⁻⁴ Mol/L, IC_{50 JJN-3}=2.33.10⁻⁵ Mol/L and IC_{50 OPM-2}=1.55×10⁻⁴ Mol/L) did not exhibit any significant difference with those of pamidronate. As IL-6 plays an important role in the proliferation of myeloma cells, we investigated the capacity of human rIL-6 (5 ng/ml) to reverse the inhibition of the HMCL proliferation. The inhibitory effects observed with the two bisphosphonates on HMCL proliferation are not reversed by the addition of 5 ng/ml of human rIL-6.

B. Zoledronate but not Pamidronate Induces Apoptosis in HMCL

To explain how these bisphosphonates induced an inhibition of HMCL proliferation, we have evaluated their capacity to induce apoptosis using APO 2.7 staining. In FIG. 2, we show that zoledronate induces a strong dose- and time-dependent apoptosis in the three HMCL, whereas pamidronate does not (data not shown). The maximum of apoptosis is observed at a concentration of 5×10⁻⁴ Mol/L after a 4-day incubation period for OPM-2 and JJN-3 and a 5-day-incubation period for LP-1. Only JJN3 is APO 2.7 labelled at the concentration of 1×10⁻⁴ Mol/L. The percentage of apoptotic cells is presented in FIG. 2.

C. Zoledronate Induces a S-Phase Blocking on Cell Cycle

When the cell cycle of LP-1 is analysed by DNA content in the presence of pamidronate, no modification of the different phases of the cell cycle is observed. On the other hand, zoledronate induces significant modifications in the cell cycle phases. An increase of the percentage of cells in S phase (60% in the zoledronate sample versus 36% in the control) associated with a decrease of the cells in G 1 phase and the emergence of a sub-G1 peak are observed. The presence of a sub-G 1 peak corresponds to the presence of apoptotic cells.

D. Pamidronate and Zoledronate Induce Apoptosis in Human BMSC.

Given that some bisphosphonates have previously been shown to induce apoptosis in HMCL as in mononuclear cells and in osteoclasts; we evaluated their effects on BMSC. We found that both pamidronate and zoledronate induced a dose dependent apoptosis of BMSC at concentration of 10 mol/L. Interestingly, pamidronate is more efficient in inducing BMSC apoptosis than zoledronate at 10⁻⁴ mol/L: pamidronate induces 97% of APO 2.7 stained cells compared to 24% of APO 2.7 positive cells for zoledronate. In the presence of 1×10⁻⁵ Mol/L of each bisphosphonate, neither pamidronate nor zoledronate is responsible for significant BMSC apoptosis. In further experiments, concentrations of bisphosphonates less than or equal to 10⁻⁶ mol/L are used in order to avoid any interference between cell viability and the effects of bisphosphonates.

E. Both Pamidronate and Zoledronate Inhibit the Constitutive Production of IL-6 by BMSC From Patients With MM.

We have investigated the effect of pamidronate and zoledronate on the constitutive production of IL-6 by BMSC obtained from long term culture of bone marrow MM samples. Six different samples of BMSC are studied. BMSC produces constitutively high levels of IL-6, ranging from 500 to 4000 pg/mL. Pamidronate significantly inhibits (40%±14% ; p=0.05) IL-6 production by BMSC. Although this inhibition is not proportional to the dose, a return to the basal level is observed with a pamidronate concentration of 10⁻¹¹ mol/L (FIG. 3). In contrast, zoledronate induces a strong inhibition of IL-6 production by BMSC and this effect is dose-dependent with maximum exhibition (60%±10%; p=0.05) obtained at a concentration of 10⁻⁶ mol/L (p=0.05). A return to the basal production of IL-6 by BMSC is observed in the presence of 10⁻⁹ mol/L of zoledronate (FIG. 3).

F. Zoledronate, but not pamidronate, inhibits IL-1β-induced interstitial collagenase (MMP-1) secretion by BMSC taken from patients with MM.

Next we investigated the effects of both pamidronate and zoledronate on the production of MMP-1 by BMSC. Six different samples of BMSC are studied with zoledronate and four of them with pamidronate in the same way. The basal secretion of MMP-1 ranged from 3 to 22 ng/ml depending on the patient sample. Among the 6 patients tested with either pamidronate or zoledronate. 5 presented a basal level below 10 ng/ml. Because the basal production of MMP-1 by BMSC is weak, the effect of bisphosphonates on MMP-1 secretion after IL-1β stimulation is analysed and a minimum five-fold MMP-1 upregulation is observed. The upregulation of MMP-1 secretion after IL-1β stimulation depends on the basal level of MMP-1 secretion and is inversely proportional to it. FIG. 4 illustrates the inhibition of MMP-1 secretion with either pamidronate or zoledronate. The addition of zoledronate significantly inhibits (44%±14%; p=0.05) MMP-1 secretion in 5 out of 6 patients and this inhibition is dose-dependent. In contrast, pamidronate has no significant effect on MMP-1 production (mean inhibition=21%±20%) and a large inter-individual variability is observed, only two out of four patient samples showing sensitivity to pamidronate. It is interesting to note that the inhibition induced by zoledronate does not depend on the basal level of MMP-1 secretion.

G. Both pamidronate and especially zoledronate enhance gelatinase A (MMP-2) expression by BMSC taken from patients with MM.

We then investigated the effects of bisphosphonates on the secretion of another matrix metalloproteinase, MMP-2 (or gelatinase A) by gelatin substrate gel zymography. MMP-2 secretion is observed in al seven patient samples studied. Both pamidronate and zoledronate increase MMP-2 production. The quantification of the 72 kDa gelatinase activity is obtained by densitometry (as shown in FIG. 5). Both pamidronate and zoledronate induce a strong upregulation at the concentration of 10⁻⁶ mol/L (165%±27% and 180%±12% with parnidronate and zoledronate respectively; p=0.05). Weak MMP-2 upregulation induced by pamidronate is observed at concentrations ranging from 10⁻⁷ to 10⁻⁹ mol/L in all patient samples tested. In contrast, zoledronate induces a strong increase of MMP-2 secretion at all concentrations ranging from 10⁻⁶ mol/L (205%±7% ) to 10⁻⁹ mol/L (159%±16%).

1. A pharmaceutical composition for treatment of malignancies which comprises in combination a bisphosphonate and.a matrix metaboproteinase inhibitor for simultaneous, sequential or separate use, provided that the bisphoshonate is not ibandronate when the MMP inhibitor is TIMP-2.

2. Use of a MMP inhibitor for the preparation of a medicament, for use in combination with a bisphosphonate for treatment of a malignant disease, for use in inhibiting MMP activity caused by the bisphosphonate, provided that the bisphosphonate is not ibandronate when the MMP inhibitor is TIMP-2.

3. A method of treating a patient suffering from a malignant disease comprising administering to the patient an effective amount of a bisphosphonate and an amount of a MMP inhibitor effective to inhibit MMP activity caused by the bisphosphonate, provided that the bisphoshonate is not ibandronate when the MMP inhibitor is TIMP-2.

4. Use of a MMP inhibitor to inhibit MMP activity caused by a bisphosphonate when the bisphosphonate is used for treatment of a malignant disease, provided that the bisphoshonate is not ibandronate when the MMP inhibitor is TIMP-2.

5. a) A pharmaceutical composition for inhibiting the development of soft tissue metastases during the treatment of a malignant disease comprising in combination a bisphosphonate and a MMP inhibitor for simultaneous, sequential or separate use, wherein treatment with the bisphosphonate gives rise to increased MMP secretion;
b) use of a MMP inhibitor in the preparation of a medicament to inhibit the development of soft tissue metastases during the treatment of a malignant disease with a combination of a bisphosphonate and a MMP inhibitor, wherein treatment with the bisphosphonate gives rise to increased MMP secretion; or
c) a method of inhibiting the development of soft tissue metastases in a patient during the treatment of a malignant disease comprising administering to the patient an effective amount of a bisphosphonate and an effective amount of a MMP inhibitor.

6. A composition according to claim 1, in which the MMP inhibitor is an inhibitor of MMP-2.

7. A composition according to claim 1 in which the bisphosphonate is pamidronate or zoledronate.
